(19) United States
(12) Patent Application Publication
(10) Pub. No.: US 2022/0088329 A1
(43) Pub. Date: Mar. 24, 2022

(54) OUTPUT DEVICE FOR A VENTILATOR

(71) Applicant: Drägerwerk AG & Co. KGaA, Lübeck (DE)

(72) Inventors: Thomas HANDZSUJ, Lübeck (DE); Doreen WERNER, Lübeck (DE); Tobias DÜSER, Lübeck (DE)

(21) Appl. No.: 17/481,462

(22) Filed: Sep. 22, 2021

(30) Foreign Application Priority Data
Sep. 23, 2020 (DE) 10 2020 124 834.2

(51) Int. Cl. A61M 16/00

(52) U.S. Cl. CPC A61M 16/0003; A61M 16/022; A61M 2205/18; A61M 2205/502; A61M 2016/0036

(57) ABSTRACT

A ventilation process and output device (100) outputs measured values of a ventilator (200). A processing unit (120) receives a first data set (122) and a second data set (126). The first data set indicates start times (123), at which a current breath (121) begins, and indicates end times (124), at which the breath ends by a starting to exhale. The second data set indicates device-side start times (127), at which the ventilator begins a current inspiratory phase (125), and indicates device-side end times (128), at which the ventilator ends the current inspiratory phase. A start deviation (140) and an end deviation (142) between the inspiratory phase of the ventilator and the current breath are determined based on the corresponding start times and the corresponding end times. An output signal (112) is provided such that within a predefined output structure (150) the start deviations and the end deviations for a predefined plurality of preceding breaths are outputted as a structured visualization (155).

### CROSS REFERENCE TO RELATED APPLICATIONS

This application claims the benefit of priority under 35 U.S.C. § 119 of German Application 10 2020 124 834.2, filed Sep. 23, 2020, the entire contents of which are incorporated herein by reference.

### TECHNICAL FIELD

The present invention pertains to an output device for outputting measured values which are provided by a ventilator and pertain to a ventilation process of the ventilator, which is connected to the person receiving medical care. The present invention pertains, furthermore, to a ventilator and to a process for outputting measured values provided by a ventilator and pertaining to a ventilation process of the ventilator, which is connected to the person receiving medical care.

### TECHNICAL BACKGROUND

In case of the implementation of different types of ventilation in ventilators, sedation devices and anesthesia devices, it is important to provide a synchronous inspiration and expiration between the patient and the ventilator. The mode of operation of a ventilation that is as synchronous as possible is typically controlled via a valve control. Switching over from inspiration to expiration is usually controlled by machine and is independent of a respective breathing effort of a patient. Against this background, for example, a higher-level pressure control acts as a safety device, so that the ventilator switches over to expiration in case of exceeding a pressure limit, for example, due to respiratory distress or coughing of the patient.

In case of pressure-assisted spontaneous breathing, the expiration valve is opened during switching over of a fixed predefined settable inspiration flow and ends the inspiration. Typically, it is true that a measured signal such as a pressure, flow or volume measured signal is detected for triggering an inspiratory ventilation stroke in case of a pressure-assisted spontaneous breathing. Minor changes in the corresponding gas flow are also measured in this case. However, these measured signals may often not guarantee perfectly synchronous coordination between the patient and the ventilator. Such an asynchrony may have negative consequences on the health of the correspondingly ventilated patient.

Against this background, it is known to output consecutive breaths of a ventilated patient via an output device and to output a value for a current asynchrony index at the same time or combined over previous breaths.

Thus, WO 2019/094736 A1 describes an output, in which the asynchrony index of a current ventilation is analyzed.

### SUMMARY

An object of the present invention is to provide an especially simple and clear display of a synchrony or asynchrony of a ventilation of a person to be ventilated.

According to a first aspect of the present invention, an output device for outputting measured values which are provided by a ventilator and pertain to a ventilation process of the ventilator, which is connected to the person receiving medical care, with an output unit and with a processing unit, is proposed according to the present invention for accomplishing this object.

The output unit is configured to receive an output signal and to provide a visual output by means of an output display screen based on the output signal.

The processing unit is configured to receive a first data set and a second data set in real time, wherein the first data set indicates personal start times, at which a respective current breath of the person begins, and indicates personal end times, at which the respective current breath of the person ends by a starting to exhale, and wherein the second data set indicates device-side start times, at which the ventilator begins with a current inspiratory phase for assisting the respective current breath of the person, and indicates device-side end times, at which the ventilator ends the current inspiratory phase. The processing unit is further configured to determine a start deviation and an end deviation between the inspiratory phase of the ventilator and the current breath of the person on the basis of the corresponding device-side and personal start times and of the corresponding device-side and personal end times.

In this case, the processing unit is further configured to provide the output signal in real time such that the start deviations and the end deviations for a predefined plurality of preceding breaths within a predefined output structure are outputted as a respective structured visualization, wherein the respective structured visualization comprises a visual display for the respective start deviation and for the respective end deviation of the respective breath, and wherein the structured visualization of the start deviation and of the end deviation of the last breath taken at a constant predefined starting position over time within the predefined output structure is outputted. In this case, chronologically preceding structured visualizations of the start deviation and the end deviation are shifted to a next position fixed within the output structure starting from this starting position, when the structured visualization of the start deviation and the end deviation of a new last breath taken at the starting position is outputted.

It was found within the framework of the present invention that for clear detection of an asynchrony between the provided ventilation and the natural breathing of the patient, a breath-specific display of this asynchrony is advantageous. Furthermore, it was found that a starting position held constant for information about the current breath supports rapid detection of the most important information from this display. Finally, the display of information about earlier breaths allows an especially reliable estimation concerning the currently present asynchrony.

The device-side and personal start times and the device-side and personal end times of the corresponding inspiratory phase are advantageously related to one another by the processing unit according to the present invention. As a result, the data for the especially clear display of the end deviation and start deviation within a structured visualization are advantageously obtained.

Moreover, the provision of the structured visualization advantageously makes possible an especially rapid and reliable estimation of the current ventilation situation with regard to the presence of an asynchrony and therefore assists the rapid discovery of a reliable treatment strategy by the user of the output device according to the present invention, i.e., preferably by a medical staff member.

The output device according to the present invention may advantageously be used for a variety of ventilators, and especially for ventilators which are already commercially available, as long as they provide the first data set and the second data set according to the present invention. The output of the first data set, which indicates the breathing activity of the person, and especially the corresponding current breath, is known. The output of the second data set, which indicates the current inspiratory phase by the ventilator, is likewise known. Therefore, the output device according to the present invention can access data sets with corresponding data which are already provided by commercially available ventilators. The structure of the corresponding ventilator and the internal data processing thereof for providing the first data set and second data set is thus likewise known to the person skilled in the art and will therefore not be described in detail below.

The starting position is a geometrically predefined position within the output structure of the visual output. The fixed next position within the output structure may be a constant, predefined position within the output structure or a predefined position which is variable over time and/or is variable based on the content of the output. The position is variable here to the effect that the corresponding next position is predefined corresponding to a predefined legitimacy. For example, an asynchrony event with a start deviation and/or with an end deviation above a deviation limit value may lead to a different fixed next position than an asynchrony event with a corresponding start deviation and/or end deviation below this deviation limit value.

The structured visualization is structured to the effect that the predefined output structure predefines fixed, predefined positions of respective information within the visual output. In this connection, the predefined output structure also comprises the arrangement and configuration of the structured visualizations of the consecutive breaths.

The output of the start deviations and end deviations by the processing unit leads according to the present invention to a visual indication, which indicates a value for the corresponding deviation.

The defined start deviations and end deviations for the predefined plurality of preceding breaths form an optically analyzable value for the currently present asynchrony between the ventilation by the ventilator and the respective breath of the person being ventilated.

Preferred embodiments of the output device according to the present invention are described below.

In an especially preferred embodiment, the fixed next position is a closest next position within the output structure. As a result, it is advantageously ensured that the most current values for the start deviations and the end deviations together with the deviations of the last breath taken in the same area of the visual output can be detected, and can especially be detected with little cognitive effort of the user. The closest next position is in this case among different possible next positions within the predefined output structure, the one which has the shortest geometric distance from the starting position. The closest next position may therefore be one of two possible closest next positions in case of two equidistant next positions.

In another advantageous embodiment, the predefined output structure is configured such that the different structured visualizations for the start deviations and end deviations of the chronologically consecutive breaths are arranged adjacent to one another and are especially arranged one below the other. Such a display of adjacent information makes possible a simple cognitive detection of the chronological sequence of received and processed data. In this case, the data, which were recorded at an especially distant point in time, are preferably moved to a corresponding next position, which is located less centrally within the output structure than the predefined starting position. As a result, it can be intuitively understood which data are the current data of the ventilator and which data were determined at an earlier time.

In another preferred embodiment, the structured visualizations are configured and arranged within the output structure such that the personal start times of consecutive breaths are arranged on a common start line, and are especially arranged directly below one another. In this embodiment, the output structure has an especially clear configuration, since all the start times of the breaths can be perceived by the user at a glance. Thus, the information, which is outputted graphically in addition to this start line, for example, are outputted in the form of a bar graph, are detected in an especially simple and rapid manner, since the start times are fixed as respective reference values on the start lines. In a preferred variant of this embodiment, the duration of the respective breath, the duration from the start deviation and/or from the end deviation is displayed in the form of a bar, of a section, of a line and/or of a graph, for example, of a graph of the other ventilation parameter, starting from the start lines. This improves the clarity of the corresponding structured visualization.

In an especially advantageous embodiment, the processing unit is configured to process the personal start time and the personal end time for the respective current breath such that a relationship between a time interval between the start time and the end time and a fixed predefined bar length of a displayed bar of the structured visualization is determined and a scaling factor corresponding to this relationship is used in order to calculate a breath-dependent scaling of the start deviation and end deviation determined and to output a correspondingly scaled start deviation and end deviation within the structured visualization. The use of the scaling factor according to this embodiment advantageously makes possible an equal dimensioning of the plurality of breaths regardless of the actual duration of the corresponding breath. Use is advantageously made here of the fact that the equal absolute deviation between mechanical ventilation and natural breathing motion of the person for a short breath represents a greater risk than for an especially long breath. Thus, for example, a greater part of the breath is worked against the ventilator in case of a short breath than this would be the case in case of a long breath with equal asynchrony, i.e., equal deviation. Furthermore, scaling allows an especially clear display of the plurality of breaths, since the displayed distance between the start and the end of a breath is equal for all breaths. A common start line, on which the personal start times are arranged, and a common end line for the end times of the breaths are used especially advantageously in a variant of this embodiment. As a result, an especially easily detectable output structure for the user of the output device can be provided.

In another advantageous embodiment, the structured visualization further comprises a display of a predefined tolerance range for the respective displayed start deviation and end deviation. Due to the display of a tolerance range, it can be rapidly detected by the user of the output device whether a current deviation, i.e., an actually present asynchrony is within or outside the predefined tolerance. Consequently, it can be rapidly detected whether this deviation requires any change in the current treatment and/or represents a risk to the health of the ventilated person. The tolerance range may in this case be marked in color or be made recognizable by a different optical marking, for example, by a frame or a shading.

In a preferred variant of the previous embodiment, the predefined tolerance range is a predefined tolerance range based on the person-specific data of the person connected to the ventilator. It can be detected in this embodiment in an especially reliable and individualized manner based on the use of the person-specific data whether the current start deviation and the end deviation, i.e., the current asynchrony of the ventilation, requires a change in the current treatment and/or represents a risk to the health of the ventilated person in view of his person-specific data.

The structured visualization further comprises in an advantageous embodiment of the output device according to the present invention a warning mark, which indicates by a visual highlighting that a predefined limit value for the start deviation and/or the end deviation is exceeded. Such a warning mark makes possible a rapid cognitive detection of the breaths from the plurality of breaths shown, in which the predefined limit value for the start deviation and/or the end deviation was exceeded. As a result, it can be especially rapidly detected whether the current treatment has to be changed, for example, whether the parameters of the mechanical ventilation by the ventilator have to be changed. The warning mark is preferably arranged directly at the current position, i.e., at the starting position or at one of the next positions within the output structure, of the corresponding display of the start deviation and end deviation. Consequently, it can be rapidly detected whether the exceeding of the predefined limit value was a single event, or whether the predefined limit value was exceeded often, i.e., whether a plurality of warning marks were outputted within the framework of the structured visualization.

In another embodiment, the output structure further comprises a display of a time curve that implies a duration of a respective breath within the displayed plurality of preceding breaths. The display of the time curve according to this embodiment is especially advantageous if a displayed length of a respective breath is standardized by the use of a corresponding scaling factor, so that the duration of the respective breath cannot be inferred based on the length. In a variant of this embodiment, the time curve is indicated by time marks in addition to at least some of the structured visualizations of a respective breath. As a result, it can be rapidly detected, over which period of time the displayed breaths of the breathing of the person to be ventilated are displayed. For example, it can be rapidly detected by the user of the output device according to the present invention whether the displayed breaths were taken in the course of the last 5 min or in the course of the last hour. The time mark or the time marks for displaying the time curve are visualized, for example, in the form of numbers, which indicate a time, a ventilation duration or another reference time. Furthermore, the number of time marks can be visualized by means of a pictographic display, for example, by a rotating hand or by a rotating tail for visualizing a clock.

In an especially preferred embodiment, the output structure further comprises a waveform display of the breathing activity of the person connected to the ventilator. In this case, the waveform display allows an association between a respective structured visualization of the start deviation and of the end deviation of a breath and the position of this breath within the waveform display. The output structure in this embodiment comprises, in addition to a visualization of the start deviation and the end deviation, the display of other features of the breathing activity of the person to be ventilated. Thus, a ventilation strategy to be carried out can especially be inferred on the basis of a maximum amplitude of a breath and on the basis of an amplitude curve on the health status of the associated person and/or on a treatment to be carried out. In a variant of this embodiment, the start deviation and the end deviation of a breath are indicated directly at the position of this breath within the waveform display, and are especially indicated over a correspondingly identified background area behind the waveform display. In an alternative or additional variant of this embodiment, the start deviation and the end deviation of the breath are outputted within the framework of the predefined output structure in addition to the entire waveform display. A visual association is preferably made between the structured visualization of the breath and the position within the waveform display by a mark of the corresponding visualization of the start deviation and of the end deviation by the user and/or by a mark of a corresponding position within the waveform display. The visual association is made, for example, by a graphic highlighting of the concrete breath within the waveform display and within the output structure of the start deviation and end deviation, for example, by the use of a predefined color or of a predefined symbol.

In an especially preferred variant of the previous embodiment, the waveform display also allows an association between a respective structured visualization of the start deviation and the end deviation of a breath and of the position of this breath within the waveform display on the basis of a common display color, and especially a dynamically variable display color. An especially rapid cognitive detection of the breath within the waveform display and within the display of the start deviation and of the end deviation is possible due to the use of the common display color.

Furthermore, the output device is preferably configured to receive preprocessed analysis data which indicate a present type of asynchrony. The output device according to the present invention is also preferably configured to output the received information regarding the types of asynchrony within the framework of the predefined output structure, especially to output this received information in addition to the start deviation and the end deviation of the breath in question. Examples of known types of asynchrony are the presence of a double trigger, i.e., that two inspiratory phases by the ventilator take place within one breath, the presence of an ineffective breathing effort, i.e., that the breathing activity of the persons does not receive any gas assistance by the ventilator, as well as the presence of a so-called Delayed Cycling, i.e., a delayed switching over of the ventilator into expiration compared with the breath of the person.

In another embodiment, the output device is configured to output an asynchrony feature within the framework of the predefined output structure. In this embodiment, it is preferably indicated at at least one position or in at least one area of the output structure that an asynchrony is present, and especially that the start deviation and/or the end deviation of at least one breath, preferably of a plurality of predefined minimum number of breaths, is above a predefined limit value.

In another embodiment, the shifting of the chronologically preceding structured visualization of the start deviation and the end deviation into the fixed next position due to a motion of the structured visualization is displayed within the framework of the visual output. Such a dynamic display which is identified by motion facilitates the visual detection of a change in the visual output, for example, of a change due to the displaying of a new last breath taken.

The output unit and the processing unit are separate from one another in one embodiment and may be configured as a common device in another embodiment. In particular, both units may access a shared processor and/or be arranged within a shared housing. The communication between the processing unit and the output unit preferably takes place in a cable-based or wireless manner.

Finally, a ventilator with an output device according to at least one of the previous embodiments is proposed according to a second aspect of the present invention for accomplishing the above-mentioned object. The ventilator has all the advantages of the corresponding embodiments of the output device according to the present invention.

The ventilator according to the present invention advantageously makes possible such a provision of the first data set and second data set that the processing unit according to the present invention can especially rapidly detect the corresponding relevant data from the two data sets. As a result, a time delay between the recording of the data by a corresponding measuring unit of the ventilator and a processing of the data by the processing unit according to the present invention is especially short.

According to a third aspect of the present invention, a process for outputting measured values which are provided by a ventilator and pertain to a ventilation process of the ventilator, which is connected to the person receiving medical care is proposed for accomplishing the above-mentioned object. The process according to the present invention has the following steps:
receipt of a first data set and of a second data set in real time, wherein the first data set indicates personal start times, at which a respective current breath of the person begins, and indicates personal end times, at which the respective current breath of the person ends by a starting to exhale, and wherein the second data set indicates device-side start times, at which the ventilator begins with a current inspiratory phase for assisting the respective current breath of the person, and indicates device-side end times, at which the ventilator ends the current inspiratory phase;
determination of a start deviation and of an end deviation between the inspiratory phase of the ventilator and the current breath of the person based on the corresponding device-side and personal start times and of the corresponding device-side and personal end times;
provision of an output signal in real time such that within a predefined output structure the start deviations and end deviations for a predefined plurality of preceding breaths are outputted as a respective structured visualization, wherein the respective structured visualization comprises a visual display for the respective start deviation and for the respective end deviation of the respective breath, and wherein the structured visualization of the start deviation and of the end deviation of the last breath taken at a constant predefined starting position over time within the predefined output structure is outputted, and wherein chronologically preceding structured visualizations of the start deviation and of the end deviation are shifted to a next position fixed within the output structure starting from this starting position, when the structured visualization of the start deviation and the end deviation of a new last breath taken at the starting position is outputted.

An especially rapid and reliable detection of the start deviations and end deviations of a sequence of breaths is made possible by the process according to the present invention. As a result, for example, during the checking of a patient, it can be rapidly detected whether the ventilation of the patient has been carried out as desired in the course of a preceding time range, for example, in the course of the last at least 5 minutes, especially at least 20 minutes, preferably at least 40 minutes. It can be especially detected whether the difficult-to-avoid asynchrony between the ventilation by the ventilator and the natural breath of the patient is in a range that is possibly critical for the patient, i.e., for example, is above a predefined limit value for the start deviation and/or end deviation of the respective breath.

An especially preferred embodiments of the process according to the present invention has the following additional steps:
processing of the personal start time and of the personal end time for the respective current breath such that a relationship between a time interval between the start time and the end time and a fixed predefined bar length of a displayed bar of the structured visualization is determined and a scaling factor corresponding to this relationship is used to calculate a breath-dependent scaling of the start deviation and end deviation determined; and
outputting of a correspondingly scaled start deviation and end deviation within the structured visualization based on the breath-dependent scaling.

The process according to this embodiment makes possible a display of the start deviation and end deviation of consecutive breaths based on the scaling factor applied, which display is especially compact and can be detected rapidly. Thus, for example, all breaths can be scaled to a uniform standardized length, as a result of which the structured display is especially clear.

The present invention shall now be explained in more detail on the basis of advantageous exemplary embodiments, which are schematically shown in the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing an exemplary embodiment of an output device according to a first aspect of the present invention;

FIG. 2 is a schematic view of an exemplary embodiment of a ventilator according to a second aspect of the present invention;

FIG. 3 is a schematic view of a first exemplary embodiment of a predefined output structure according to the present invention;

FIG. 4 is a schematic view of a second exemplary embodiment of the predefined output structure according to the present invention;

FIG. 5 is a schematic view of a third exemplary embodiment of the predefined output structure according to the present invention;

FIG. 6 is a schematic view of a fourth exemplary embodiment of the predefined output structure according to the present invention;

FIG. 7 is a schematic view of a fifth exemplary embodiment of the predefined output structure according to the present invention; and

FIG. 8 is a flow chart of an exemplary embodiment of a process according to a third aspect of the present invention.

### DESCRIPTION OF PREFERRED EMBODIMENTS

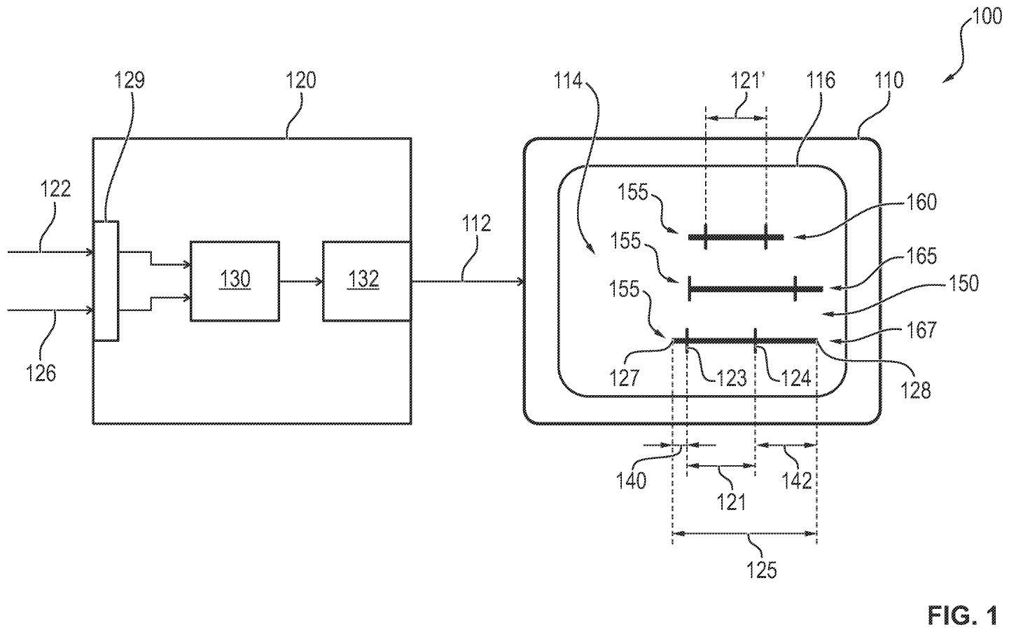

Referring to the drawings, FIG. 1 shows a schematic view of a first exemplary embodiment of an output device 100 according to a first aspect of the present invention.

The output device 100 is configured for outputting measured values that are provided by a ventilator and pertain to a ventilation process of the ventilator, which is connected to the person receiving medical care. In this case, the output device 100 has an output unit 110 and a processing unit 120.

The output unit 110 is configured to receive an output signal 112 and to provide a visual output 114 by means of an output display screen 116 based on the output signal 112. The output unit 110 has a corresponding output signal interface for receiving the output signal 112. This may be an interface for a cable-based or a wireless connection to the processing unit 120. Possible embodiments of such interfaces are known to the person skilled in the art and will therefore not be further explained below.

The processing unit 120 is configured to receive a first data set 122 and a second data set 126 in real time. In real time in this case means that current data are processed and as a result only a slight time delay exists between the determination (acquisition) of the data and the receipt of the data (at the processor), especially a time delay of less than 5 sec, preferably of less than 2 sec, especially preferably of less than 1 sec. The two data sets are received by at least one data set interface 129. In the exemplary embodiment being shown, the two data sets are received individually by precisely one data set interface. In an exemplary embodiment, not shown, the data sets are received by two separate data set interfaces. Such a data set interface is configured for a cable-based or wireless receipt of the two data sets 122, 126. The first data set 122 indicates personal start times 123, at which a respective current breath 121 of the person begins, and personal end times 124, at which the respective current breath 121 of the person ends by a starting to exhale. The second data set 126 indicates device-side start times 127, at which the ventilator for assisting the respective current breath 121 of the person begins with a current inspiratory phase 125, and device-side end times 128, at which the ventilator ends the current inspiratory phase 125. A start deviation 140 and an end deviation 142 between the inspiratory phase 125 of the ventilator and the current breath 121 of the person is determined by the processing unit 120 in a corresponding deviation determination module 130 on the basis of the corresponding device-side and personal start times 123, 127 and of the corresponding device-side and personal end times 124, 128. For the marked breath, the start deviation 140 is markedly smaller than the end deviation 142, which means that the inspiratory phase 125 has started almost synchronously with the breath 121, whereas the inspiratory phase 125 by the ventilator was ended markedly later than the breath 121.

In addition to the deviation determination module 130, the processing unit 130 comprises an output signal determination module 132, which is configured to provide the output signal 112 in real time. In this case, the provision in real time means that there is only a small time delay, especially a time delay of less than 5 sec, preferably of less than 2 sec, and especially preferably of less than 1 sec between the receipt of the two data sets 122, 126 and the output of the output signal 112. In this case, the output signal 112 is provided such that the start deviations 140 and the end deviations 142 are outputted within a predefined output structure 150 for a predefined plurality of preceding breaths 121 as a respective structured visualization 155. The structured visualizations 155 within the predefined output structure 150 can be seen based on the visual output 114 on the output display screen 116 of the output unit 110 shown in FIG. 1. The structured visualization 155 comprises a visual display for the respective start deviation 140 and for the respective end deviation 142 of the corresponding breath 121. In the exemplary embodiment shown, the visual display is carried out by a bar graph. The personal start times 123 and the personal end times 124 of a breath 121 are in this case displayed by means of corresponding marks on a bar of the bar graph indicating the inspiratory phase 125.

The structured visualization 155 of the start deviation 123 and of the end deviation 124 of the last breath taken 121′ is outputted at a constant predefined starting position over time 160 within the predefined output structure 150. The predefined starting position 160 in the exemplary embodiment being shown is the position for the structured visualization 155, which is arranged at the upper edge of the display screen of the output display screen 116. From the upper display screen edge to the lower display screen edge of the output display screen 116, three breaths 121, 121′ are outputted in the form of a respective structured visualization 155, wherein the last breath taken is arranged in the starting position 160 at the upper display screen edge and the breath taken beforehand is arranged at the correspondingly fixed next position 165, and the, in turn, next breath is arranged at the other fixed next position 167. In this case, a respective fixed next position 165, 167 within the predefined output structure is a closest next position 165, 167, i.e., a next position, which has the shortest geometric distance from the present next positions. Thus, the additional fixed next position 167 is at a farther distance than the fixed next position 165. Due to such a sequence of next positions for the structured visualization 155 of an original breath, it is ensured that the chronological sequence of the respective breaths 121, 121′ can be detected by a user of the output device 100 rapidly and reliably.

Starting from this starting position 160, chronologically preceding structured visualizations 155 of the start deviation 140 and the end deviation 142 are shifted to the next positions 165, 167 described when the structured visualization 155 of the start deviation 140 and the end deviation 142 of a new last breath taken 121′ at the starting position is outputted. The shifting is preferably dynamically displayed by means of a motion of the corresponding structured visualization 155, so that a user of the output device 100 detects the output of a new last breath taken 121′.

In the exemplary embodiment shown, the output unit 110 and the processing unit 120 are separate devices, which are connected to one another in a cable-based manner, for example, via an Ethernet cable. In an exemplary embodiment, not shown, these two devices are connected in a wireless manner, for example, via a WLAN connection, via a Bluetooth connection, via a BLE connection or via a ZigBee connection.

According to the present invention, the output unit and the processing unit may form a common device, which is controlled by a shared processor. In particular, the output unit and the processing unit are arranged in a common housing.

The shown modules of the processing unit 120 are preferably actuated by a shared processor. The modules are in this case preferably processing units, which can be distinguished from each other at least one a software level, for processing the two data sets 122, 126.

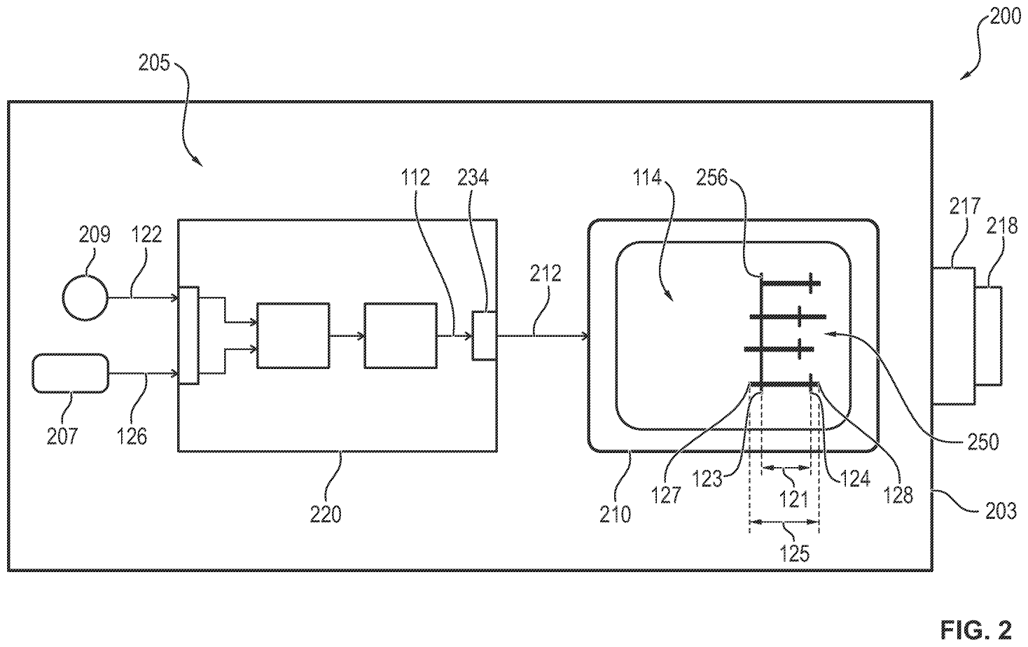

FIG. 2 shows a schematic view of an exemplary embodiment of a ventilator 200 according to a second aspect of the present invention.

The ventilator 200 comprises the output device 205 according to the present invention, which differs from the output device 100 shown in FIG. 1 only in that the processing unit 220 has, furthermore, an output module 234, which is configured to receive the output signal 112 of the deviation determination module 130 and to convert the output signal 112 into a readable output signal 212, wherein the readable output signal 212 is a signal that can be read by the output unit 210. Furthermore, the output device 205 differs from the output device 110 in that the output unit 210 has an input unit 217 with a user interface 218, via which the user can control the visual output 114. The user interface 218 is in the exemplary embodiment being shown a number of buttons. The user interface is a setting wheel, a keyboard, a switch, a touchpad and/or a touch display in an exemplary embodiment, not shown.

It can be seen within the framework of the visual output 114 that the predefined output structure 250 provides that all personal start times 123 of consecutive breaths 121 are arranged on a common start line 256, and are especially arranged directly below one another.

In the exemplary embodiment shown, the output unit 210 and the processing unit 220 are arranged within a common housing 203 of the ventilator 200. The ventilator 200 further comprises an internal reading unit 207 in order to read the device-side start times 127 and the device-side end times 128 of the inspiratory phase 125 provided by the ventilator 200 and to provide the corresponding second data set 126. Finally, the ventilator 200 comprises a measuring unit 209, which measures the personal start times 123 and end times 124 of a respective current breath 121 of the person ventilated by the ventilator 200 and is configured to provide the first data set 122. The precise configuration of such a measuring unit 209 is known to the person skilled in the art from commercially available ventilators and will therefore not be explained in detail below.

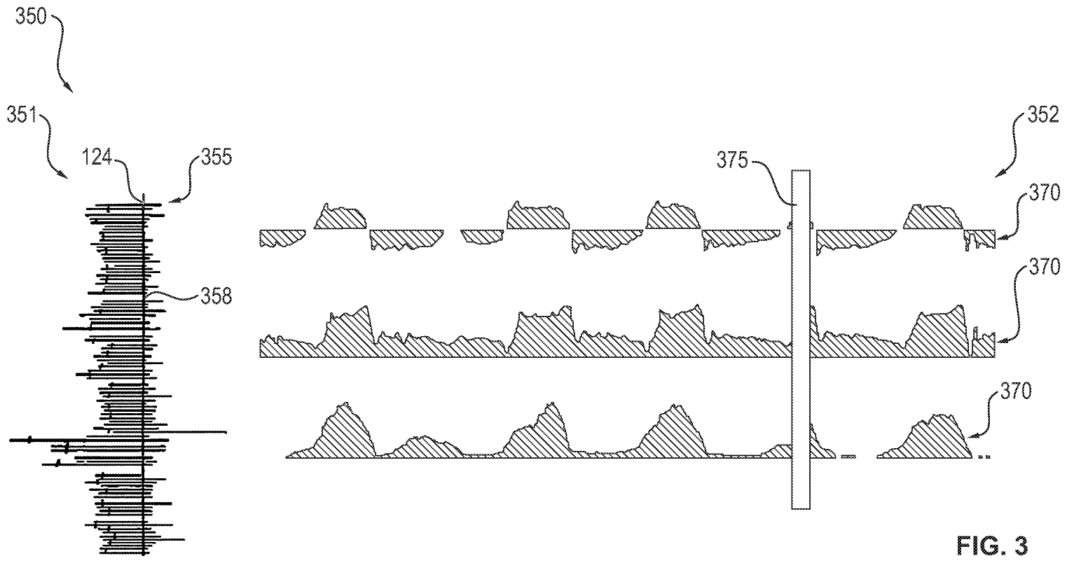

FIG. 3 shows a schematic view of a first exemplary embodiment of a predefined output structure 350 according to the present invention.

The predefined output structure 350 comprises a first area 351, in which a plurality of structured visualizations 355 are outputted below one another from an upper area of the visual output to a lower area of the visual output. In this case, the personal end times 124 of a respective breath are directly below one another on a common end line 358.

The predefined output structure 350 further comprises a second area 352, in which three different waveform displays 370 of physiological parameters of the person connected to the ventilator are displayed. At least one of the waveform displays 370 describes a breathing activity of the person connected to the ventilator. A white bar 375 within the three different waveform displays 370 indicates at what point of the display current measured values, which pertain to the breathing of the person, are added. In this case, the white bar 375 is typically moved by the waveform displays 370 such that the respective waveform display is kept static and an overwriting of all measured values of an earlier measurement by current measured values is illustrated only by the movable bar.

By means of the white bar 375, the waveform display 370 makes possible an association between the respective structured visualization 355 of the start deviation and end deviation of a breath and the position of this breath within the waveform display 370. Thus, the last breath, which is shown in front of the white bar 378, is the breath, which is displayed at the topmost point of the predefined output structure 350 in the first area 351 in the form of a structure visualization 355.

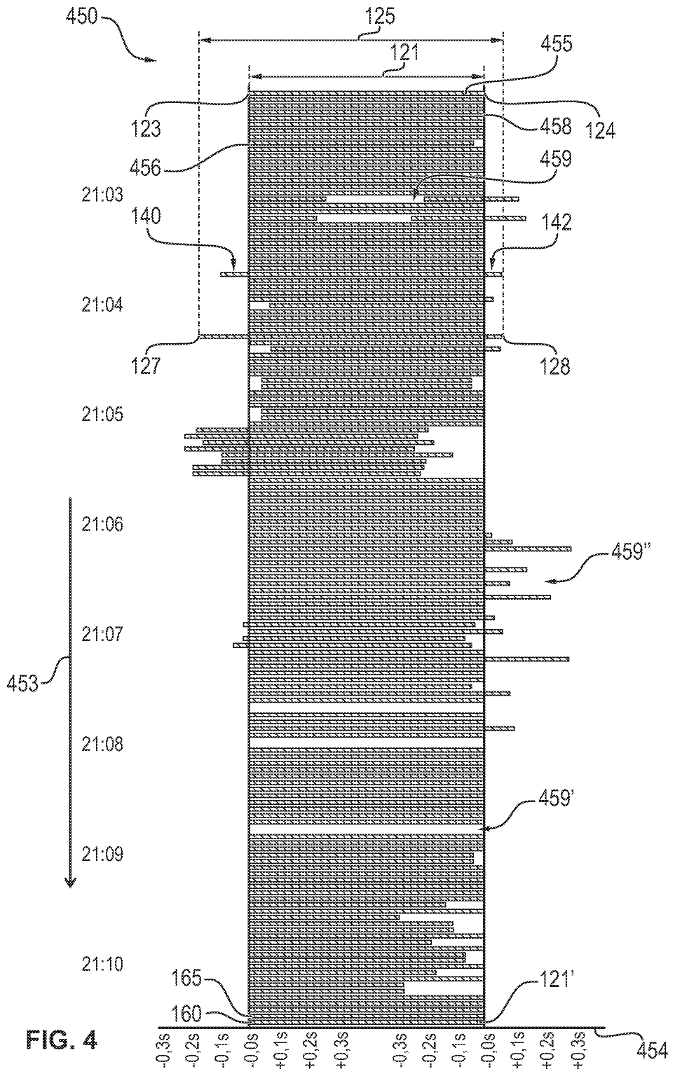

FIG. 4 shows a schematic view of a second exemplary embodiment of the predefined output structure 450 according to the present invention.

Within the framework of the output structure 450, all structured visualizations of a respective breath are scaled such that personal start times 123 and personal end times 124 lead to a respective equal bar length for the respective breath 121. In this case, all personal start times are arranged directly below one another on a common start line 456. Moreover, all personal end times 214 are arranged on a common end line 458.

The respective inspiratory phases 125 are scaled with the same scaling factor such as the length of the breath 121. As a result, the device-side start times 127 and the device-side end times 128 are not standardized to a uniform length in their display.

The starting position 160 for a last breath taken 121′ is arranged at the lower edge of the predefined output structure 450. The preceding breaths 121 have been shifted upwards from the starting position 160 above the respective closest fixed next position 165. Along the vertical axis 453 of the predefined output structure 450, a current time to the minute is indicated after each minute, so that an overall course duration for the display course of breaths can be rapidly detected by the user of the corresponding output device. Such a display of the time implies a time curve (time course) of the breaths, in the sense that a duration of a respective breath is implied. A percentage deviation is indicated along a horizontal axis 454 for the correspondingly respective start deviations 140 and end deviations 142 displayed within a structured visualization. In this connection, the displayed percentage deviation indicates, for example, in the exemplary embodiment shown, what percentage 452 of an overall duration of the respective breath are the present start deviation 140 and the present end deviation 142. Should a respective deviation not be present, then the personal start time 123 and the device-side start time 127 are arranged together on the start line 456 and/or the personal end time 124 and the device-side end time 128 are arranged on the end line 458.

Furthermore, three different types of asynchronicity within the consecutive structured visualizations 455 can be seen in FIG. 4. Two different double trigger events are shown in a first asynchronicity position 459. A double trigger event is characterized in that two inspiratory phases 125 are carried out by the corresponding ventilator within one breath 121. Therefore, the person to be ventilated experiences no breathing assist by the ventilator for a certain period of time within the breath. A plurality of ineffective breathing efforts are shown in a second asynchronicity position 459′. An ineffective breathing effort is characterized in that no inspiratory phase is carried out by means of the ventilator within a breath, so that the person receives no gas assist, especially no pressure, flow, anesthesia or sedation gas assist for the spontaneous breathing activity. Hence, the person has to carry out the complete breath without assistance by the ventilator. A plurality of delayed cycling events are shown in a third asynchronicity position 459″. A delayed cycling event is characterized in that a switching over of the ventilator from inspiration to expiration is delayed. Therefore, device-side gas is being fed even after the end of the breath, since the device-side end times of the inspiratory phase are only behind the personal end time of the breath.

The corresponding output device in the exemplary embodiment being shown is advantageously configured to output the presence of an asynchronicity event, especially to output same by means of a corresponding mark within the predefined output structure 450. It can preferably be detected on the basis of the corresponding mark which asynchronicity event it is. The information about which asynchronicity event has occurred is preferably outputted by a corresponding, predefined pictographic display, which is, however, not shown in the present case.

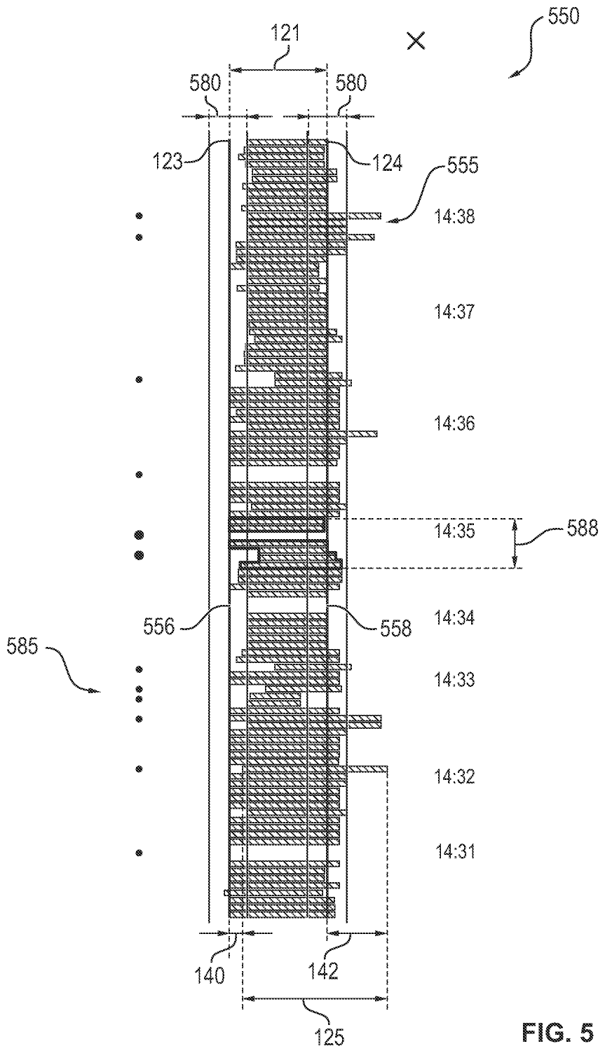

FIG. 5 shows a schematic view of a third exemplary embodiment of the predefined output structure 550 according to the present invention.

The output structure 550 differs from the output structure 450 shown in FIG. 4 by no horizontal axis being provided with percentage data for estimating the start deviations 10 and end deviations 142. Instead of this, each structured visualization 555 has a display of a predefined tolerance range 580 in the respective area of the start line 556 and in the respective area of the end line 558. In the exemplary embodiment being shown, the tolerance range 580 comprises an area of 15% of the entire duration of the respective breath 121 before and after the respective personal start time 123 and the respective personal end time 124. This tolerance range is in an exemplary embodiment, not shown, displayed as lighter than the area outside of the respective tolerance range in order to make possible a simple and rapid visual detection.

In the exemplary embodiment being shown, the tolerance range 580 for each patient comprises a range of 15% of the entire duration of the respective breath 121. In an exemplary embodiment, not shown, the predefined tolerance range is based on person-specific data of the person connected to the ventilator. Thus, for example, a different tolerance range may be useful from a medical point of view for a person with severely damaged lungs than for a person with an only slightly limited lung function.

Furthermore, the output structure 550 differs from the output structure shown in FIG. 4 in that the structured visualization 555 comprises a respective position, in which a warning mark 585 is shown, as soon as a predefined limit value for the start deviation 140 and/or for the end deviation 142 for the concrete breath 121 with the associated inspiratory phase 125 is exceeded. The predefined limit value is in the exemplary embodiment being shown equal to the fixed tolerance range 580 of 15% of the entire duration of the respective breath 121. Due to the visual display of the tolerance range 580 with the corresponding inspiratory phases, the reason why the warning mark 585 was triggered can be immediately seen by the user of the output device according to this exemplary embodiment. Thus, it can be directly detected whether a too early start of the inspiratory phase 125, a too late start of the inspiratory phase 125, a too early end of the inspiratory phase 125, a too late end of the inspiratory phase 125 or the like is present.

The warning mark 585 is in the exemplary embodiment shown a dot, which is arranged at a spaced location from the structured visualization 555 of the breath, is shown on the left-hand side of the corresponding bar graph.

Furthermore, it is possible within the framework of the output structure 550 shown to select a time range 588 of breaths by means of a corresponding user interface and to receive for this range other physiological and/or patient-specific information, not shown in FIG. 5, such as, for example, at least one measured result of an additional sensor connected to the patient to be ventilated. The time range 588 is shown in the exemplary embodiment being shown by a light background and/or by a box structure around the time range 588, which can be seen.

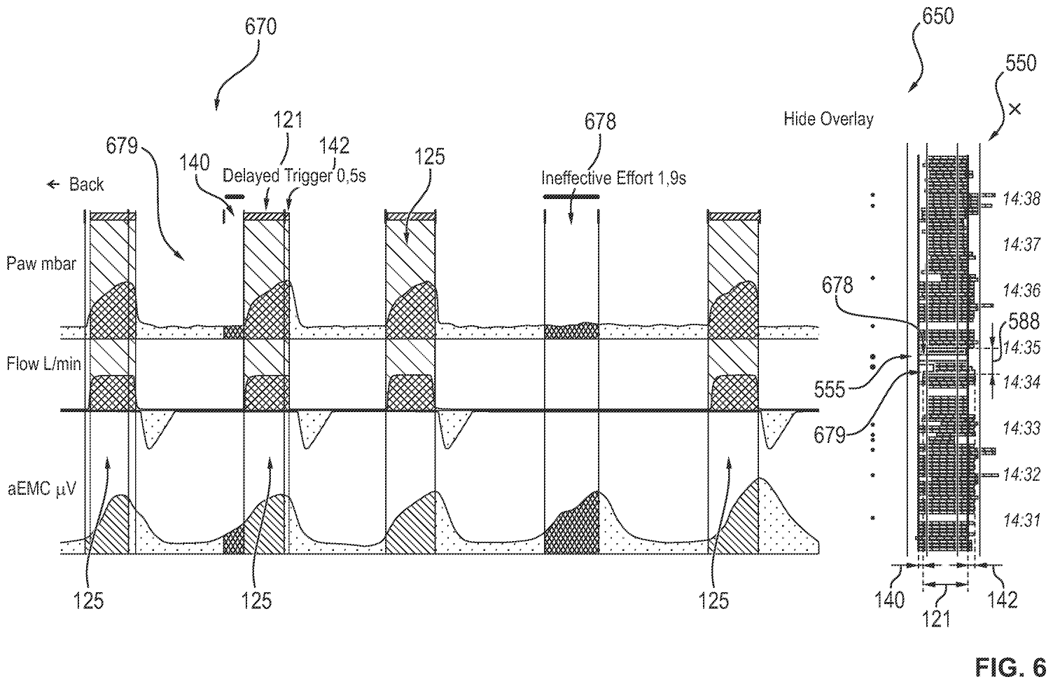

FIG. 6 shows a schematic view of a fourth exemplary embodiment of the predefined output structure 650 according to the present invention.

The output structure 650 comprises the output structure 550 shown in FIG. 5, wherein the output structure 650 further comprises a number of waveform displays 670. The waveform displays 670 comprise especially a display of the breathing activity of the person connected to the ventilator. An association between a respective structure visualization 555 of the start deviation 140 and of the end deviation 142 of a breath 121 and the position of this breath 121 within a respective waveform display 670 is carried out here by means of the selection of the time range 588 and the display of the respective physiological measured values within the waveform displays 670, which were recorded within the selected time range 588.

Consequently, the analyzed time range 588 can be rapidly changed by means of an interaction with a corresponding user interface and a breathing activity of the person to be ventilated for a preceding time can thereby be investigated or checked.

In an especially preferred variant, not shown, a selected breath is indicated by graphic highlighting, especially by highlighting in color, within a waveform display and as a result allows an association between the structured visualization of the start deviation and end deviation and the corresponding position of this breath within the waveform display. The highlighting in color is carried out, for example, via a display color, which differs from the display of the other data, and especially via a dynamically variable display color, which visualizes, for example, a time curve through its dynamic change. Thus, a predefined difference in shade or in a used gray scale between the selected breath and other breath areas may correspond to a predefined distance over time.

In the exemplary embodiment being shown, the respective inspiratory phases 125 are marked in color within the waveform display 670 and thereby allow a rapid visual detection of the relationship between the personal breath 121 and the device-side inspiratory phase 125. The personal start times 123 and the personal end times 124 are each shown as a black line by all waveforms in order to be consistent with the bar display of the structured visualizations 655. Asynchrony events are marked as respective surfaces over all waveforms in a way that is different from the mark of the inspiratory phases, in which the respective tolerance range 580 is not exceeded. For example, the asynchrony events are displayed in a different color, with a different shade, with a border of a different color and/or with a different color combination or with a different color contrast.

In the exemplary embodiment shown, an ineffective breathing effort 678 and a so-called delayed trigger 679, i.e., a too late start of the inspiratory phase, within the selected time range 588 are displayed as asynchrony events.

In a preferred variant of the exemplary embodiment shown, the displayed time range 588 can be changed dynamically by a corresponding user interface, especially can be changed by a scrolling of a user interface configured as a setting wheel, so that the corresponding waveform display 670 is shifted dynamically with the selected time range 588 by the interaction with the user interface.

In the exemplary embodiment shown, the waveform displays 670 are three physiological measured curves, especially a pressure curve, a gas flow curve and an electromyographic respiratory muscle activity curve (sEMG curve) in the present case.

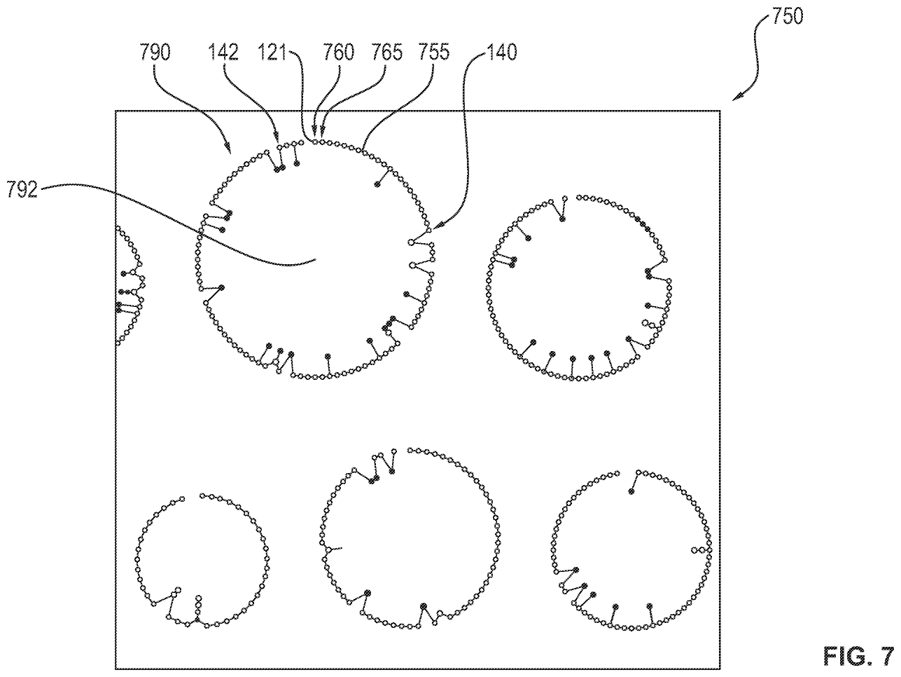

FIG. 7 shows a schematic view of a fifth exemplary embodiment of the predefined output structure 750 according to the present invention.

The output structure 750 differs from the output structures of the previous exemplary embodiments in that the respective structured visualizations 755 of the start deviations 140 and the end deviations 142 are not arranged along a horizontal or vertical section, but rather along a circle circumference 790. The start deviation 140 and the end deviation 142 can in this case each be detected by a shift of a dot out of the circle circumference 790. The shift may in this case take place in the direction towards the corresponding circle center 792 or away from this circle center 792.

In the exemplary embodiment being shown, a start deviation 140 and an end deviation 142 are indicated only if they exceed a predefined limit value. In this case, all deviations are displayed as a shift of a dot, which is on the circle circumference 790 for breaths without exceeding the predefined limit value, in a direction towards the corresponding circle center 792. The type of the present asynchrony event is apparent for the user of the corresponding output device by the manner of the display of the corresponding dot, for example, the shape thereof, the shading thereof, the color thereof and/or the magnitude thereof. The amplitude of the shift of the dot is in this case a value of the magnitude of the start deviation 140 and/or of the end deviation 142.

As shown in FIG. 7, the circular display allows a clear presentation of a plurality of ventilations, especially a presentation of the ventilation of a plurality of persons to be ventilated. In this case, it can be individually specified for each person over which preceding period of time the breaths shall be displayed as corresponding dots. Correspondingly, the displayed circle circumferences 790 differ in their respective size, i.e., in the number of the breaths displayed.

In the exemplary embodiment shown, the starting position 760 for displaying the last breath taken is the topmost dot of the respective circle circumference 790, from where the structured visualization 755 of the respective breath 121 is shifted clockwise from next position to next position 765. The shift into the respective next position is carried out counterclockwise in an exemplary embodiment, not shown.

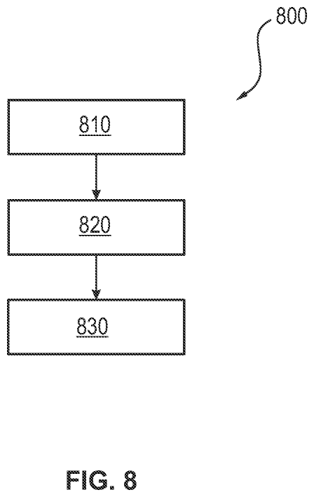

FIG. 8 shows a flow chart of an exemplary embodiment of a process 800 according to a third aspect of the present invention.

The process 800 according to the present invention is configured for outputting measured values which are provided by a ventilator and pertain to a ventilation process of the ventilator, which is connected to the person receiving medical care. In this case, the process 800 has the steps explained below.

A first step 810 comprises a receipt of a first data set and a second data set in real time. In this case, the first data set indicates personal start times, at which a respective current breath of the person begins, and personal end times, at which the respective current breath of the person ends by a starting to exhale. Furthermore, the second data set indicates device-side start times, at which the ventilator begins with a current inspiratory phase for assisting the respective current breath of the person, and device-side end times, at which the ventilator ends the current inspiratory phase.

A next step 820 comprises a determination of a start deviation and of an end deviation between the inspiratory phase of the ventilator and the current breath of the person based on the corresponding device-side and personal start times and on the corresponding device-side and personal end times.

Finally, a final step 830 comprises a provision of an output signal in real time such that within a predefined output structure, the start deviations and the end deviations for a predefined plurality of preceding breaths are outputted as a respective structured visualization, wherein the respective structured visualization comprises a visual display for the respective start deviation and for the respective end deviation of the respective breath. The structured visualization of the start deviation and of the end deviation of the last breath taken at a constant predefined starting position over time within the predefined output structure is outputted in this case, wherein chronologically preceding structured visualizations of the start deviation and end deviation are shifted to a next position fixed within the output structure starting from this starting position, when the structured visualization of the start deviation and end deviation of a new last breath taken at the starting position is outputted.

Steps 810, 820 and 830 are carried out in this order according to the present invention. Thus, the two data sets are first received, then the respective start deviation and end deviation for a respective breath with a respective associated inspiratory phase are determined and the output signal according to the present invention is then generated and provided in real time.

Provision in real time means according to the present invention that there is a minimal, preferably barely perceptible time delay between the receipt of the two data sets and the provision of the output signal, especially a time delay of less than 5 sec, preferably a time delay of less than 2 sec, and especially preferably a time delay of less than 1 sec.

In an especially preferred exemplary embodiment, a final step, which comprises a receipt of the output signal and a provision of a visual output based on the output signal, is added to the process 800 according to the present invention.

While specific embodiments of the invention have been shown and described in detail to illustrate the application of the principles of the invention, it will be understood that the invention may be embodied otherwise without departing from such principles.

### LIST OF REFERENCE NUMBERS

100, 205 Output device
110, 210 Output unit
112 Output signal
114 Visual output
116 Output display screen
120, 220 Processing unit
121 Breath
121′ Last breath taken
122 First data set
123 Personal start time
124 Personal end time
125 Inspiratory phase
126 Second data set
127 Device-side start time
128 Device-side end time
129 Data set interface
130 Deviation determination module
132 Output signal determination module
140 Start deviation
142 End deviation
150, 250, 350, 450, 550 Predefined output structure
650, 750
155, 355, 455, 555, 755 Structured visualization
160, 760 Starting position
165, 167, 765 Next position
200 Ventilator
203 Housing
207 Reading unit
209 Measuring unit
212 Readable output signal
217 Input unit
218 User interface
234 Output module
256, 456, 556 Start line
358, 458, 558 End line
351 First area
352 Second area
370, 670 Waveform displays
375 White bar
453 Vertical axis
454 Horizontal axis
459, 459′, 459″ Asynchrony position
580 Tolerance range
585 Warning mark
588 Selected time range
678 Ineffective breathing effort
679 Delayed trigger event
790 Circle circumference
792 Circle center
800 Process
810, 820, 830 Process steps

What is claimed is:

1. An output device for outputting measured values, which are provided by a ventilator and pertain to a ventilation process of the ventilator connected to a person receiving medical care, the output device comprising:
an output unit configured to receive an output signal and to provide a visual output by means of an output display screen based on the output signal; and
a processing unit configured:
to receive a first data set and a second data set in real time, wherein the first data set indicates personal start times, at which a respective current breath of the person begins, and indicates personal end times, at which the respective current breath of the person ends by a starting to exhale, and the second data set indicates device-side start times, at which the ventilator begins with a current inspiratory phase for assisting the respective current breath of the person, and indicates device-side end times, at which the ventilator ends the current inspiratory phase;
to determine a start deviation and an end deviation between the inspiratory phase of the ventilator and the current breath of the person based on the corresponding device-side start times and personal start times and the corresponding device-side end times and personal end times;
to provide the output signal in real time such that the start deviations and the end deviations for a predefined plurality of breaths within a predefined output structure are outputted as a respective structured visualization, which respective structured visualization comprises a visual display for the respective start deviation and for the respective end deviation of the respective breaths, and the structured visualization of the start deviation and of the end deviation of a last breath taken at a constant predefined starting position over time within the predefined output structure is outputted, and chronologically preceding structured visualizations of the start deviation and of the end deviation are shifted to a next position fixed within the output structure starting from the starting position, when the structured visualization of the start deviation and of the end deviation of a new last breath taken at the starting position is outputted.

2. An output device in accordance with claim 1, wherein the next position, which is fixed, is a closest next position within the output structure.

3. An output device in accordance with claim 1, wherein the predefined output structure is configured such that the structured visualization of the start deviation and of the end deviation of the breaths, which breaths are chronologically consecutive are arranged adjacent to one another.

4. An output device in accordance with claim 3, wherein the structured visualization of the start deviation and of the end deviation of the breaths are configured and arranged within the output structure such that the personal start times of consecutive breaths are arranged on a common start line.

5. An output device in accordance with claim 1, wherein the processing unit is further configured to process the personal start time and personal end time, for the respective current breath, such that a relationship between a time interval between the start time and the end time and a fixed predefined bar length of a displayed bar of the structured visualization is determined and a scaling factor, corresponding to the relationship is used to calculate a breath-dependent scaling of the start deviation and end deviation determined and to output a correspondingly scaled start deviation and end deviation within the structured visualization.

6. An output device in accordance with claim 1, wherein the structured visualization further comprises a display of a predefined tolerance range for the respective displayed start deviation and end deviation.

7. An output device in accordance with claim 6, wherein the predefined tolerance range is a based on person-specific data of the person connected to the ventilator.

8. An output device in accordance with claim 1, wherein the structured visualization further comprises a warning mark, which indicates, by a visual highlighting, that a predefined limit value for the start deviation and/or the end deviation is exceeded.

9. A ventilator comprising an output device for outputting measured values which pertain to a ventilation process of the ventilator connected to a person receiving medical care, the output device comprising:
an output unit configured to receive an output signal and to provide a visual output by means of an output display screen based on the output signal; and
a processing unit configured:
to receive a first data set and a second data set in real time, wherein the first data set indicates personal start times, at which a respective current breath of the person begins, and indicates personal end times, at which the respective current breath of the person ends by a starting to exhale, and the second data set indicates device-side start times, at which the ventilator begins with a current inspiratory phase for assisting the respective current breath of the person, and indicates device-side end times, at which the ventilator ends the current inspiratory phase;
to determine a start deviation and an end deviation between the inspiratory phase of the ventilator and the current breath of the person based on the corresponding device-side start times and personal start times and the corresponding device-side end times and personal end times;
to provide the output signal in real time such that the start deviations and the end deviations for a predefined plurality of breaths within a predefined output structure are outputted as a respective structured visualization, which respective structured visualization comprises a visual display for the respective start deviation and for the respective end deviation of the respective breaths, and the structured visualization of the start deviation and of the end deviation of a last breath taken at a constant predefined starting position over time within the predefined output structure is outputted, and chronologically preceding structured visualizations of the start deviation and of the end deviation are shifted to a next position fixed within the output structure starting from the starting position, when the structured visualization of the start deviation and of the end deviation of a new last breath taken at the starting position is outputted.

10. A ventilator in accordance with claim 9, wherein the next position, which is fixed, is a closest next position within the output structure.

11. A ventilator in accordance with claim 9, wherein the predefined output structure is configured such that the structured visualization of the start deviation and of the end deviation of the breaths, which breaths are chronologically consecutive are arranged adjacent to one another.

12. A ventilator in accordance with claim 11, wherein the structured visualization of the start deviation and of the end deviation of the breaths are configured and arranged within the output structure such that the personal start times of consecutive breaths are arranged on a common start line.

13. A ventilator in accordance with claim 9, wherein the processing unit is further configured to process the personal start time and personal end time, for the respective current breath, such that a relationship between a time interval between the start time and the end time and a fixed predefined bar length of a displayed bar of the structured visualization is determined and a scaling factor, corresponding to the relationship is used to calculate a breath-dependent scaling of the start deviation and end deviation determined and to output a correspondingly scaled start deviation and end deviation within the structured visualization.

14. A ventilator in accordance with claim 9, wherein the structured visualization further comprises a display of a predefined tolerance range for the respective displayed start deviation and end deviation.

15. A ventilator in accordance with claim 14, wherein the predefined tolerance range is a based on person-specific data of the person connected to the ventilator.

16. A ventilator in accordance with claim 9, wherein the structured visualization further comprises a warning mark, which indicates, by a visual highlighting, that a predefined limit value for the start deviation and/or the end deviation is exceeded.

17. A process for outputting measured values which are provided by a ventilator and pertain to a ventilation process of the ventilator, which is connected to a person receiving medical care, the process comprising the steps of:
receiving a first data set and a second data set in real time, wherein the first data set indicates personal start times, at which a respective current breath of the person begins, and indicates personal end times, at which the respective current breath of the person ends by a starting to exhale and the second data set indicates device-side start times, at which the ventilator begins with a current inspiratory phase for assisting the respective current breath of the person, and indicates device-side end times, at which the ventilator ends the current inspiratory phase;
determining a start deviation and an end deviation between the inspiratory phase of the ventilator and the current breath of the person based on the corresponding device-side and personal start times and on the corresponding device-side and personal end times;
providing an output signal in real time such that within a predefined output structure the start deviations and end deviations for a predefined plurality of breaths are outputted as a respective structured visualization, wherein the respective structured visualization comprises a visual display for the respective start deviation and for the respective end deviation of the respective breaths, the structured visualization of the start deviation and of the end deviation of the last breath taken at a constant predefined starting position over time within the predefined output structure is outputted, and chronologically preceding structured visualizations of the start deviation and of the end deviation are shifted to a next position fixed within the output structure starting from this starting position, upon the structured visualization of the start deviation and of the end deviation of a new last breath taken at the starting position is outputted.

18. A process in accordance with claim 17, further comprising the steps of:
processing the personal start time and the personal end time for the respective current breath such that a relationship between a time interval between the start time and end time and a fixed predefined bar length of a displayed bar of the structured visualization is determined and a scaling factor corresponding to the relationship is used to calculate a breath-dependent scaling of the determined start deviation and end deviation;
outputting a correspondingly scaled start deviation and end deviation within the structured visualization based on the breath-dependent scaling.
